(19) United States
(12) Patent Application Publication
(10) Pub. No.: US 2009/0187246 A1
(43) Pub. Date: Jul. 23, 2009

(54) Interbody implants for spinal alignment procedures

(76) Inventor: Kevin T. Foley, Germantown, TN (US)

Correspondence Address: MEDTRONIC;Attn: Noreen Johnson - IP Legal Department, 2600 Sofamor Danek Drive, MEMPHIS, TN, 38132, US

(21) Appl. No.: 12/009,658

(22) Filed: Jan. 22, 2008

(51) Int. Cl. A61F 2/44

(52) U.S. Cl. 623/17.16; 623/17.11

(57) ABSTRACT

Spinal implants are provided that include a body having opposite vertebral bearing surfaces. One of the bearing surfaces includes engaging means to engage an adjacent vertebra and the other of the bearing surfaces provides a substantially smooth surface profile to permit another of the adjacent vertebra to be moved along and in contact with the smooth bearing surface as corrective forces are applied to manipulate the other of the adjacent vertebrae into alignment.

### BACKGROUND

Misalignment of one or more vertebrae along the spinal column is corrected by moving the vertebrae into an aligned position through application of correct forces that re-orient or re-position one vertebra relative to another vertebra until the desired alignment is achieved. Implants positioned in the space between vertebral bodies can hinder application of the corrective forces since the implants can engage the adjacent vertebrae in a manner that resists movement of the vertebrae relative to the implant, hindering alignment of the vertebrae. If the resistance is overcome, the implant may become misaligned or moved out of the desired position in the disc space as the vertebrae are aligned. There remains a need for interbody spinal implants that can be effectively employed in procedures for correcting spinal alignment.

### SUMMARY

Spinal implants are provided that include a body having opposite bearing surfaces. One of the bearing surfaces includes engaging means to engage one of the adjacent vertebrae and the other of the bearing surfaces provides a smooth surface profile to permit the other of the adjacent vertebrae to be moved along the smooth bearing surface as corrective forces are applied to manipulate the other of the adjacent vertebrae into alignment.

According to another aspect, a spinal implant comprises a body sized for positioning in a spinal disc space between adjacent vertebrae. The body includes a first bearing surface and an opposite second bearing surface extending along the body. The body further includes a height between the first and second bearing surfaces that provides a restored disc space height when positioned in the spinal disc space. The first and second bearing surfaces define a respective entire side of the body and the sides are positionable in contact with respective ones of the adjacent vertebra. The first bearing surface is entirely smooth and the second bearing surface includes means for engaging the respective adjacent vertebra.

According to another aspect, a method for correcting alignment of a spinal column, comprises: positioning a spinal implant in a disc space between first and second vertebrae; fixing the spinal implant in position with the first vertebra; and sliding the second vertebra along a smooth bearing surface of the spinal implant to align the first and second vertebrae.

According to another aspect, a spinal implant comprises a body sized for positioning in a spinal disc space between adjacent vertebrae. The body extends along an axis between a distal leading end and a proximal trailing end. The body further includes sidewalls extending between the leading end and the trailing end. The body also includes a first bearing surface extending along a first side of the body between the sidewalls and the leading end and the trailing end. The first bearing surface is entirely smooth where it contacts one of the adjacent vertebrae. The body also includes a second bearing surface that extends along a second side of the body between the sidewalls and the leading end and the trailing end. The second bearing surface includes engaging features extending therefrom for engaging the other of the adjacent vertebrae.

These and other aspects will be discussed further below.

### BRIEF DESCRIPTION OF THE DRAWINGS

FIG. 1 is a side elevation view of a misaligned spinal column segment.

FIG. 2 is a side elevation view of the misaligned spinal column segment with a spinal implant in a disc space between vertebrae and the vertebrae in partial section to show contact of the spinal implant with the adjacent vertebrae.

FIG. 3 is a side elevation view of the spinal column segment in an aligned condition with the vertebrae in partial section to show contact of the spinal implant with the adjacent vertebrae.

FIG. 4 is a side elevation view of the aligned spinal column segment with the vertebrae in partial section to show contact with the spinal implant and with a stabilization construct engaged to the adjacent vertebrae.

FIG. 5A is a top plan view of an inferior vertebra of the spinal column segment with a pair of spinal implants positioned therein in a posterior approach to the disc space.

FIG. 5B is a perspective view looking toward the inferior bearing surface of the spinal implant in FIG. 5A.

FIG. 6A is a top plan view of the inferior vertebra of the spinal column segment with a spinal implant positioned therein in an anterior approach to the disc space.

FIG. 6B is a side elevation view of the spinal implant of FIG. 6A.

FIG. 6C is an enlarged view showing one embodiment of engaging features on the inferior bearing surface of the spinal implant of FIG. 6B.

FIG. 7A is a top plan view of the inferior vertebra of the spinal column segment with another embodiment spinal implant positioned therein in an anterior approach to the disc space.

FIG. 7B is a side elevation view of the spinal implant of FIG. 7A.

FIG. 7C is a perspective view of the spinal implant of FIG. 7B rotated 180 degrees to look down on the inferior bearing surface.

FIG. 8A is a top plan view of the inferior vertebra of the spinal column segment with a pair of spinal implants positioned therein in an anterior approach to the disc space.

FIG. 8B is a perspective view looking toward the proximal end of one of the spinal implants of FIG. 8A.

FIG. 9A is a top plan view of the inferior vertebra of the spinal column segment with a spinal implant positioned therein in a lateral approach to the disc space.

FIG. 9B is a side elevation view of the spinal implant of FIG. 9A.

FIG. 9C is an end elevation view of the spinal implant of FIG. 9B showing another arrangement for the engaging features on the inferior bearing surface of the spinal implant.

FIG. 10A is a top plan view of the inferior vertebra of the spinal column segment with a spinal implant positioned therein in a postero-lateral approach to the disc space.

FIG. 10B is a top plan view of the spinal implant of FIG. 10A.

FIG. 10C is a bottom plan view of the spinal implant of FIG. 10A.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Spinal implants are provided that include a body having opposite vertebral bearing surfaces. One of the bearing surfaces includes engaging means to engage one of the adjacent vertebrae and the other of the bearing surfaces provides a smooth surface profile to permit the other of the adjacent vertebrae to be moved along the smooth bearing surface in contact with the implant as corrective forces are applied to manipulate one or more of the adjacent vertebrae into alignment.

The spinal implants discussed herein are employed in spinal stabilization procedures. In one procedure, the axial and/or rotational alignment of one or more vertebrae is adjusted after the implant is positioned in the spinal disc space between adjacent vertebrae. The spinal implant has a height between the bearing surfaces that provides a desired disc space height when inserted in the disc space. The spinal implant positively engages one of the vertebrae with engaging features from one of the bearing surfaces, typically the inferior vertebra, to fix the implant to the vertebra and maintain the relative positioning of the spinal implant and the vertebra. The bearing surface of the implant opposite the bearing surface with the engaging features, typically the superior bearing surface, provides a smooth surface profile so that the other vertebra can slide along smooth bearing surface of the implant while maintaining contact with the implant until the desired spinal alignment is achieved. Since the disc space height is restored prior to alignment of the vertebrae, it is not necessary to distract the vertebrae after alignment. Proper alignment of the vertebrae is more readily attained since the vertebrae are aligned with the disc space height restored. One or more stabilization constructs can be positioned along and engaged to the one or more aligned vertebral levels to maintain the corrected alignment post-operatively.

Various configurations for the smooth bearing surface are contemplated. For example, in one form the smooth bearing surface is solid and entirely free of pits, depressions, pores, indentations, recesses, or other formations so that there is no interruption in the smooth bearing surface to increase the frictional resistance of the smooth bearing surface and inhibit sliding of the adjacent bone along the smooth bearing surface. In another form, all or a portion of the smooth bearing surface is comprised of porous material and/or includes pores, pits, depressions, or indentations that extend into the smooth surface but are sufficiently small in size or otherwise configured so that the smooth bearing surface does not positively engage nor provide substantial resistance when the adjacent bone is positioned in contact therewith and slid or repositioned along the smooth bearing surface. Furthermore, it is contemplated that the smooth bearing surface can be interrupted by one or more openings to provide avenues for bone growth into the implant. In other embodiments, the implant is solid and the smooth bearing surface includes no openings or interruptions.

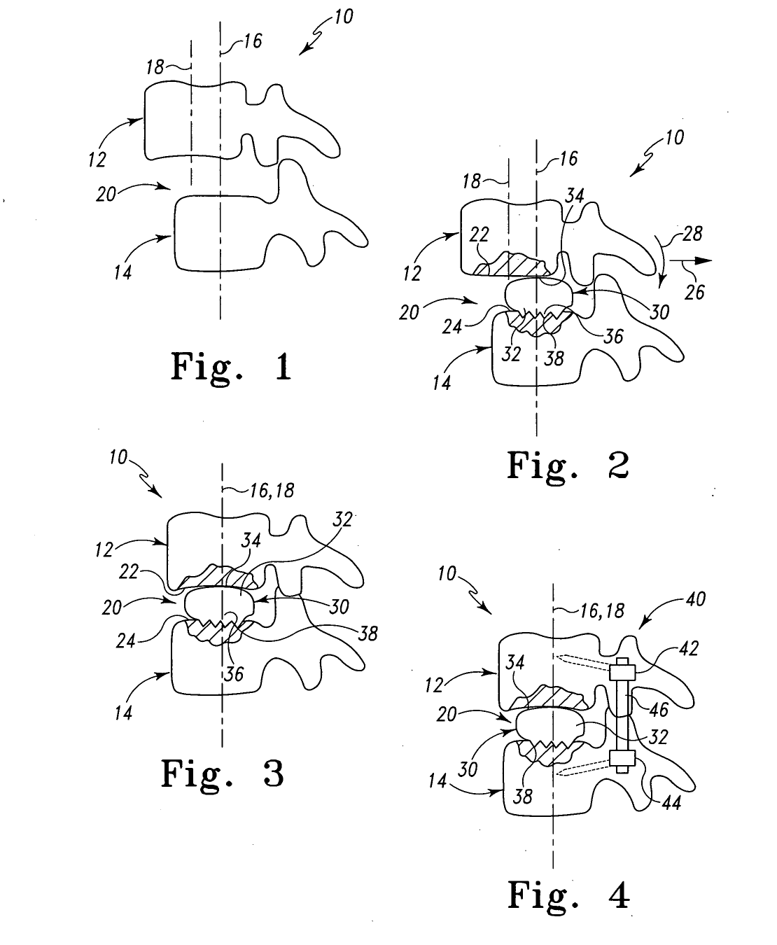

In FIG. 1 there is shown a spinal column segment 10 that includes a first vertebra 12 and a second vertebra 14. First vertebra 12 is located superiorly or cephaladly relative to the inferior or caudally located vertebra 14. Spinal column segment 10 includes a central axis 16 that corresponds to the central axis of the spinal column. First vertebra 12 is out of alignment relative to second vertebra 14 such that vertebral body axis 18 is offset from or obliquely oriented to the central spinal column axis 16 extending through vertebra 12.

In FIG. 2 an interbody spinal implant 30 is positioned in the disc space 20 located between vertebrae 12, 14. Spinal implant 30 includes a body 32 with a superior or upper bearing surface 34 positioned along an inferior surface 22 of first vertebra 12. Body 32 also includes an inferior or lower bearing surface 36 that is positioned along the superior surface 24 of second vertebra 14. Inferior surface 22 and superior surface 24 of vertebrae 12, 14 are the vertebral endplates of the respective vertebrae 12, 14 in one embodiment. In another embodiment, one or both of the surfaces 22, 24 are formed by removing bone material from the respective vertebrae 12, 14. In still other embodiments, surfaces 12, 14 are adjacent surfaces of other portions of vertebrae 12, 14, such as adjacent surfaces of the spinous processes.

Spinal implant 30 includes engaging features 38 extending from inferior bearing surface 36 that extend into or positively engage the bone of second vertebra 14 adjacent superior surface 24 and engage the second vertebra 14 to resist displacement of spinal implant 30 relative to second vertebra 14. Superior bearing surface 34 defines a surface area that forms an entire side of implant 30 that is positioned in contact with first vertebra 12, and the entire surface area is smooth so that inferior surface 22 can slide and translate along superior bearing surface 34 when corrective forces are applied to first vertebra 12.

After positioning spinal implant 30 in disc space 20, one or more corrective or reduction forces 26, 28 are applied to first vertebra 12 to improve the alignment of vertebra axis 18 relative to central axis 16 of the spinal column. For example, a translational corrective force 26 displaces first vertebra 12 such that inferior surface 22 and first vertebra 12 move generally parallel to the axial plane of the spinal column along and in contact with superior bearing surface 34. Movement in the axial plane is shown such that vertebra 12 moves posteriorly from the position of FIG. 2 to the corrected position of FIG. 3. In other procedures, corrective axial forces are applied to move first vertebra 12 in the axial plane laterally, anteriorly or obliquely relative to the sagittal plane. Rotational corrective force 28 can also or alternatively be applied to pivot and translate vertebra 12 along superior bearing surface 34 and relative to second vertebra 14 to more closely align vertebra axis 18 with central axis 16. The rotational forces can be applied in the sagittal plane as shown in FIG. 2. Rotational forces can also be applied in the coronal plane, the axial plane, or in any plane between the sagittal, coronal and axial planes.

In any of the procedures, first vertebra 12 can slide along the superior bearing surface 34 of implant 30 even with superior bearing surface 34 remaining in contact with implant 30 during the movement. First vertebra 12 is positioned to more closely align or align vertebra axis 18 with central axis 16 of the spinal column, as shown in FIG. 3. Engaging features 38 positively engage second vertebra 14 so that spinal implant 30 remains in position relative to second vertebra 14 and the other spinal structures such as the spinal canal during manipulation of first vertebra 12. Spinal implant 30 provides a height between surfaces 34, 36 that restores and/or maintains a desired disc space height and separation between vertebrae 12, 14 during alignment of the vertebrae 12, 14.

Spinal stabilization systems can be engaged to first and second vertebrae 12, 14 to maintain the corrected positioning of first vertebra 12. For example, FIG. 4 shows first and second anchors 42, 44 engaged to pedicles of respective ones of the first and second vertebrae 12, 14. A stabilization element 46 extends between and is coupled to the anchors 42, 44 to maintain the corrected vertebral alignment along spinal column segment 10. The spinal stabilization system 40 is configured to extend along one or more adjacent vertebral levels to spinal column segment 10. Stabilization element 46 can be an elongated spinal rod, plate, tether, cable, or other device. Stabilization element 46 can be rigid or flexible. Stabilization element 46 can be configured to resist tension loading only, or tension and compression loading. In the illustrated embodiment, stabilization element 46 is engaged posteriorly to the posterior vertebral elements such as the pedicles. It is further contemplated a second stabilization construct can be secured to the spinal column segment 10 on the contra-lateral side of the vertebrae 12, 14. In other procedures, one or more stabilization constructs are engaged along one or more vertebral levels along the lateral, antero-lateral and/or anterior sides of the spinal column.

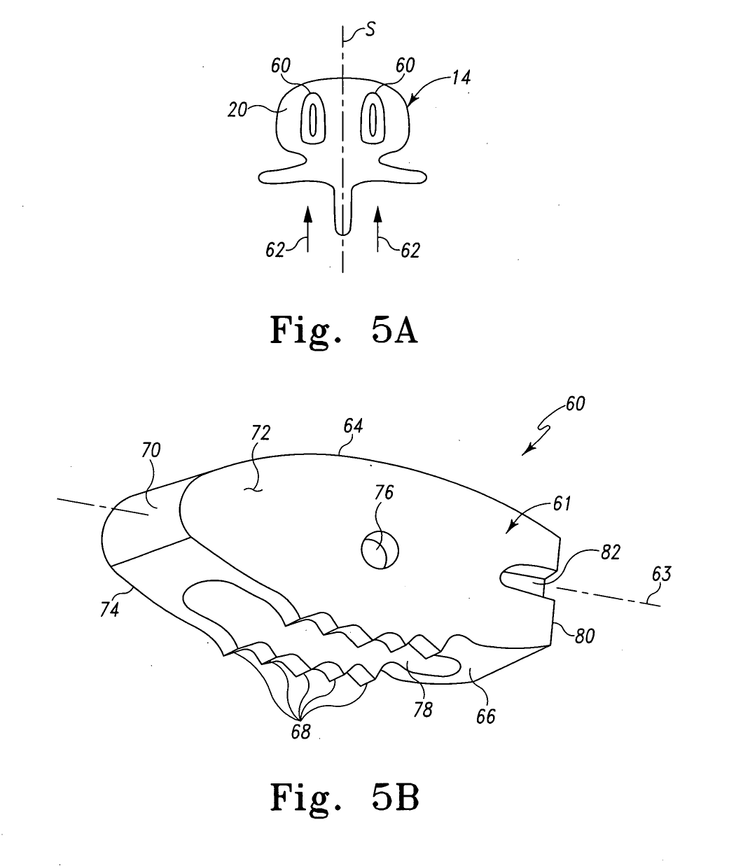

Various insertional techniques and configurations for spinal implant 30 are contemplated. For example, in FIG. 5A there is shown a top plan view of second vertebra 14 with an embodiment of spinal implant 30, designated as spinal implant 60, positioned in disc space 20. Spinal implant 60 is shown in further detail in FIG. 5B. A pair of spinal implants 60 are arranged in side-by-side relation in disc space 20 and in generally parallel relative to sagittal plane S. Spinal implants 60 are positioned in disc space 20 from a posterior approach offset on respective lateral sides of sagittal plan S, as indicated by arrows 62. When viewed in the axial plane of the spinal column, implants 60 each define a generally rectangular shape with a rounded nose. Other embodiments contemplate other shapes in the axial plane, including conical, frusto-conical, and non-rectangular shapes.

Spinal implant 60 is shown in further detail in FIG. 5B, and includes an elongated body 61 extending along a longitudinal axis 63. Body 61 includes a superior bearing surface 64 that is smooth, and an inferior bearing surface 66 that includes engaging features in the form of teeth 68. Teeth 68 extend along a middle portion of inferior bearing surface 66 that is located along the opening of cavity 78 in bearing surface 66. Other embodiments contemplate teeth on the entire area of inferior bearing surface 66. Teeth 68 are V-shaped with grooves between adjacent teeth to receive bone and resist movement of implant 60 relative to the bone engaged by teeth 68. Other embodiments contemplate other configurations for the engaging features, including spikes, pyramidal shapes, irregular shapes, elongated ridges, teeth or ridges with flattened areas, and teeth with a ratcheting configuration, for example. Still other embodiments contemplate engaging features in the form of knurlings, surface roughenings, or surface etchings, for example.

Body 61 further includes a rounded leading or distal end nose 70 that is convexly curved between superior and inferior bearing surfaces 64, 66. End nose 70 can also be convexly curved between opposite sidewalls 72, 74. The end nose 70 facilitates insertion of the implant 60 and recapitulation of the spinal disc space 20 as implant 60 is inserted therein by distracting vertebrae 12, 14. In the implanted orientation of FIG. 5A, end nose 70 is located anteriorly in disc space 20. Other procedures contemplate implant 60 is inserted so that end nose 70 is located posteriorly or laterally in disc space 20 when implant 60 is implanted.

Sidewalls 72, 74 extend parallel to longitudinal axis 63, and include one or more openings, such as shown with opening 76, that are in communication with a central cavity 78. Central cavity 78 opens at superior and inferior bearing surfaces 64, 66, and can receive bone growth material to allow fusion of the adjacent vertebrae through cavity 78. Spinal implant 60 also includes a proximal or trailing end wall 80 extending transversely to longitudinal axis 63. Proximal end wall 80 can include recesses opening therein that extend along each of the sidewalls 72, 74, such as is shown with recess 82, to engage an insertion instrument. In addition to or alternatively to recesses 82, any other suitable structure or configuration for engagement by an insertion tool is contemplated, including one or more grooves, slots and/or holes in proximal end wall 80 that are threaded or unthreaded. Further examples of spinal implants and insertion techniques are discussed in U.S. Patent Application Publication No. U.S. 2004/0162616 published on Aug. 19, 2004, which is incorporated herein by reference.

After positioning spinal implants 60 in disc space 20, corrective forces can be applied to align the superior vertebra 12 with the central axis of the spinal column, as discussed above. The superior vertebra slides, rotates and translates along superior bearing surface 64 while teeth 68 positively engage inferior vertebra 14 to maintain the positioning of spinal implants 60 relative to inferior vertebra 14 as superior vertebra 12 is re-positioned. In other procedures, only one spinal implant 60 is positioned in disc space 20, and the contra-lateral side of the disc space has no spinal implant. After correction of the vertebral alignment, the contra-lateral side can remain without an implant, or any other suitable spinal implant can be positioned therein.

FIG. 6A shows inferior vertebra 14 and disc space 20 with another embodiment of spinal implant 30, designated as spinal implant 90, positioned in disc space 20. Spinal implant 90 includes a size and shape that occupies substantially all the disc space 20 and supports the adjacent vertebrae 12, 14 at or adjacent their cortical rims about the perimeter of the respective endplate of the vertebrae 12, 14. Spinal implant 90 is positioned in disc space 20 from an anterior approach to disc space 20, as indicated by arrow 92. When viewed in the axial plane of the spinal column, implant 90 includes a kidney bean type shape.

Spinal implant 90 includes a body 91 extending along a central axis 93 aligned along sagittal plane S in the implanted position. Body 91 defines a central cavity 112 bordered by an anterior wall 104 and a posterior wall 106. Anterior and posterior walls 104, 106 are connected by convexly curved sidewalls 108, 110. Anterior wall 104 is convexly curved away from central cavity 112, and posterior wall 106 is concavely curved toward central cavity 112. Other embodiments contemplate that one of anterior and posterior walls 104, 106 is linear. In still other embodiments, anterior and posterior walls 104, 106 are both linear, include linear and curved sections, or include complex curvatures. In yet another embodiment, spinal implant 90 is provided without a central cavity 112.

As further shown in FIG. 6B, a side elevation view of spinal implant 90, the walls 104, 106, 108, 110 define a smooth superior bearing surface 94 and an opposite inferior bearing surface 96 that includes engaging features in the form of teeth 98 extending therefrom. Superior and inferior bearing surfaces 94, 96 are convexly curved to provide an intimate fit with the adjacent concavely curved surfaces of the respective vertebrae 12, 14. In the illustrated embodiment, the height of body 91 between bearing surfaces 94, 96 is tapered from a first height at anterior wall 104 that is greater than a second height at posterior wall 106. In another embodiment, one or both of the superior and inferior bearing surfaces 94, 96 are planar. In still another embodiment, superior and inferior bearing surfaces 94, 96 are parallel to one another, or are configured so that the height at anterior wall 104 is substantially the same as the height at posterior wall 106. In yet another embodiment, one or both of the superior and inferior bearing surfaces 94, 96 forms a wall that covers cavity 112 and includes openings to permit bone growth therethrough.

In FIG. 6C an enlarged detail view of teeth 98 is provided that shows one configuration, it being understood that other configurations for the engaging features could be provided as discussed herein with respect any of the other embodiments. Each of the teeth 98 includes a rectangular or square base 114 and an upper sloped portion 116 that forms an elongated V-shaped outer end 118. The teeth are formed in an array on inferior bearing surface 96 and separated by rows and columns to allow teeth 98 to penetrate and engage inferior vertebra 14. The proximal wall can include any of the insertion tool engaging features discussed with respect to the other embodiments herein.

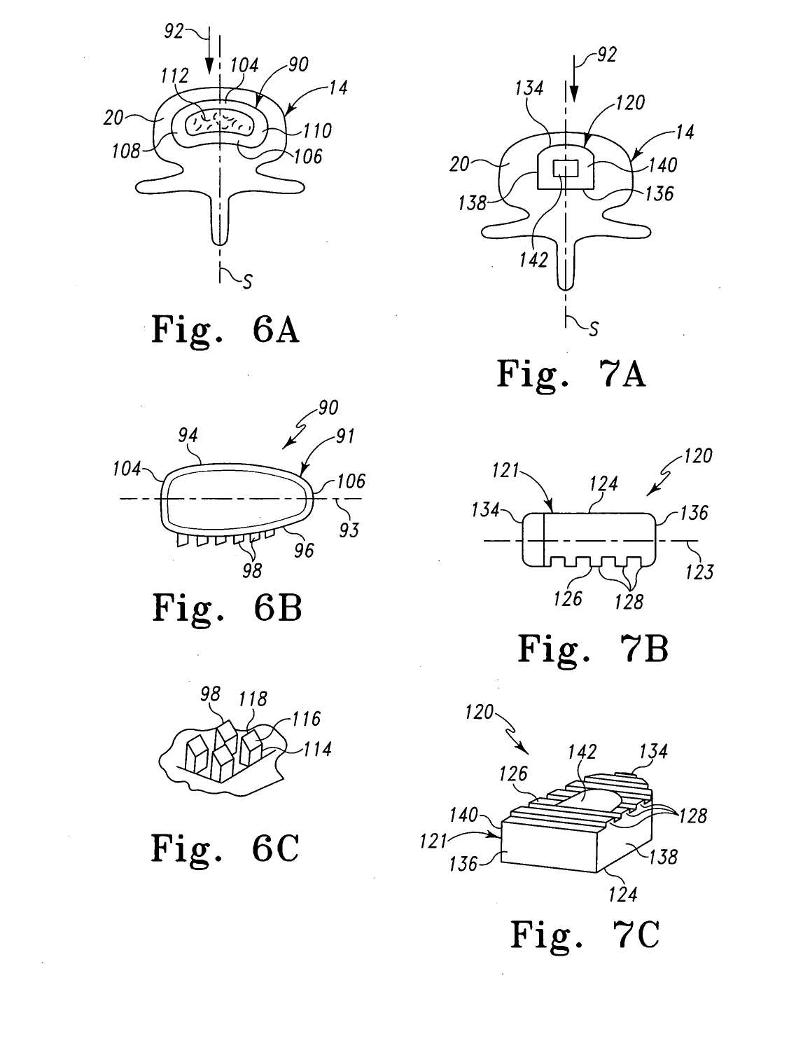

FIG. 7A shows inferior vertebra 14 and disc space 20 with another embodiment of spinal implant 30, designated as spinal implant 120, positioned in disc space 20. Spinal implant 120 includes a size and shape that occupies a substantial portion of disc space 20 and supports the adjacent vertebrae at the anterior and posterior portions of their cortical rims. Spinal implant 120 is positioned in disc space 20 from an anterior approach to disc space 20, as indicated by arrow 92. When viewed in the axial plane of the spinal column, spinal implant 120 includes a D-shape.

Spinal implant 120 includes a body 121 extending along a central axis 123 aligned along sagittal plane S in the implanted orientation. Body 121 defines a central cavity 142 bordered by an anterior wall 134 and a posterior wall 136. Anterior and posterior walls 134, 136 are connected by linear and parallel sidewalls 138, 140. Anterior wall 134 is convexly curved away from central cavity 142, and posterior wall 136 is linear between sidewalls 138, 140. Other embodiments contemplate that anterior wall 134 is linear. In yet another embodiment, spinal implant 120 is provided without a central cavity 142.

As further shown in FIG. 7B, a side elevation view of spinal implant 120, and in FIG. 7C, a perspective view with the inferior bearing surface oriented upwardly, the height of body 121 is constant from anterior wall 134 to posterior wall 136. Other embodiments contemplate a configuration that tapers in height anteriorly or posteriorly. The walls 134, 136, 138, 140 define a smooth superior bearing surface 124 and an opposite inferior bearing surface 126 that includes engaging features in the form of ridges 128 extending across body 121. Superior and inferior bearing surfaces 124, 126 are planar in the illustrated embodiment. Other embodiments contemplate that bearing surfaces 124, 126 are convexly curved to provide an intimate fit with the adjacent concavely curved surfaces of the respective vertebrae 12, 14. In yet another embodiment, one or both of the superior and inferior bearing surfaces 124, 126 forms a wall that covers cavity 142 and includes openings to permit bone growth therethrough.

Ridges 128 are shown with a rectangular or square configuration formed by rectangular slots or grooves extending across inferior bearing surface 126 between opposite sides of body 121. Other embodiments contemplate ridges that are V-shaped and/or that are ratcheted to facilitate insertion while providing greater resistance to movement back along the insertion path. In still other embodiments, the engaging features are teeth, or include any other configurations for the engaging features as discussed herein with respect any of the other embodiments. The proximal wall can include any of the insertion tool engaging features discussed with respect to the other embodiments herein.

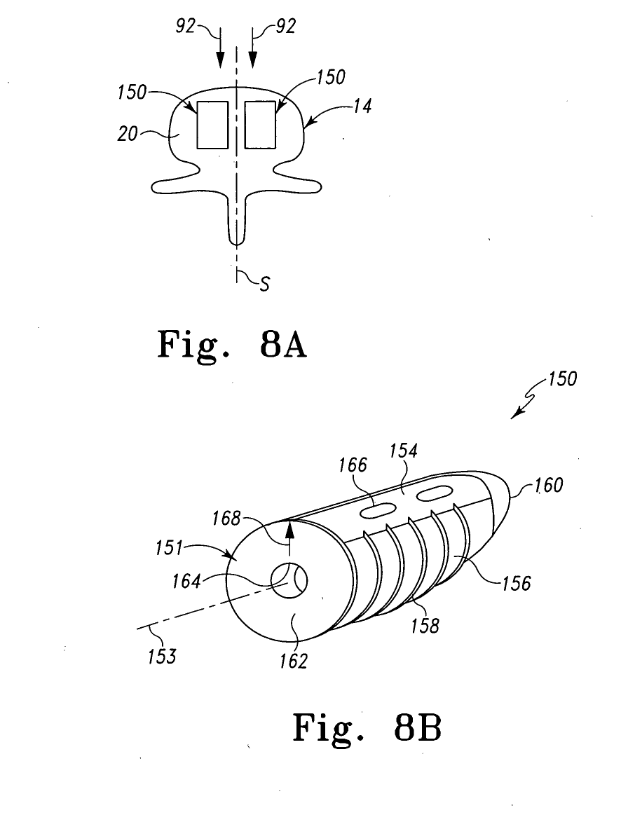

FIG. 8A shows inferior vertebra 14 and disc space 20 with another embodiment of spinal implant 30, designated as spinal implant 150, positioned in disc space 20. Spinal implant 150 includes a size and shape that occupies a portion of disc space 20 such that a pair of spinal implants 150 can be positioned in side-by-side relation in disc space 20 and provide bi-lateral support of the adjacent vertebrae. Spinal implants 150 are positioned in disc space 20 from an anterior approach to disc space 20, as indicated by arrow 92, wherein the respective approaches are offset on opposite sides of sagittal plane S. In other procedures it is contemplated that a single implant 150 is positioned on one side of sagittal plane S, or that a single implant 150 is positioned along sagittal plane S. When viewed in the axial plane of the spinal column, implant 150 defines a rectangular shape. In another embodiment, sides of implant 150 are tapered to define a frusto-conical shape in the axial plane and/or sagittal plane.

As further shown in FIG. 8B, spinal implant 150 includes a body 151 extending along a longitudinal axis 153 between a distal leading end 160 and a proximal trailing end 162. Body 151 can define a circular or substantial portion of a circular shape when viewed in the direction of longitudinal axis 153. A portion of body 151 extending along longitudinal axis 153 is smooth to provide a superior bearing surface 154 with a smooth surface, while all or a part of the remaining portion of body 151 along longitudinal axis 153 includes engaging features in the form of threads 158 extending from at least an inferior bearing surface 156. Body 151 can define a central chamber or cavity, and includes one or more holes 166 extending therethrough to permit bone growth into the cavity.

Spinal implant 150 is inserted by threading it along the respective vertebrae 12, 14 into disc space 20 until leading end 160 is positioned at the desired depth in the disc space and superior bearing surface 154 is oriented in contact with the respective adjacent surface of superior vertebra 12. Threads 158 engage the inferior vertebra 14 to maintain the positioning of spinal implant 150 relative thereto as the superior vertebra 12 is moved along superior surface 154 to the desired orientation. Proximal end 162 can include an insertion tool engaging feature 164 such as a threaded hole as shown, or include any one or combination of slots, holes, and sidewall recess to engagement by an insertion instrument. Proximal end 164 can be provided with an indicator such as a mark or arrow 168 to provide an indication of the orientation of superior bearing surface 154 relative to the vertebrae 12, 14, facilitating the surgeon in attaining the proper alignment of spinal implant 150 in situ. Leading end 160 can be in the form of a rounded nose with a bullet-shape as shown to facilitate insertion between vertebrae 12, 14. Other embodiments contemplate a leading end without a rounded nose.

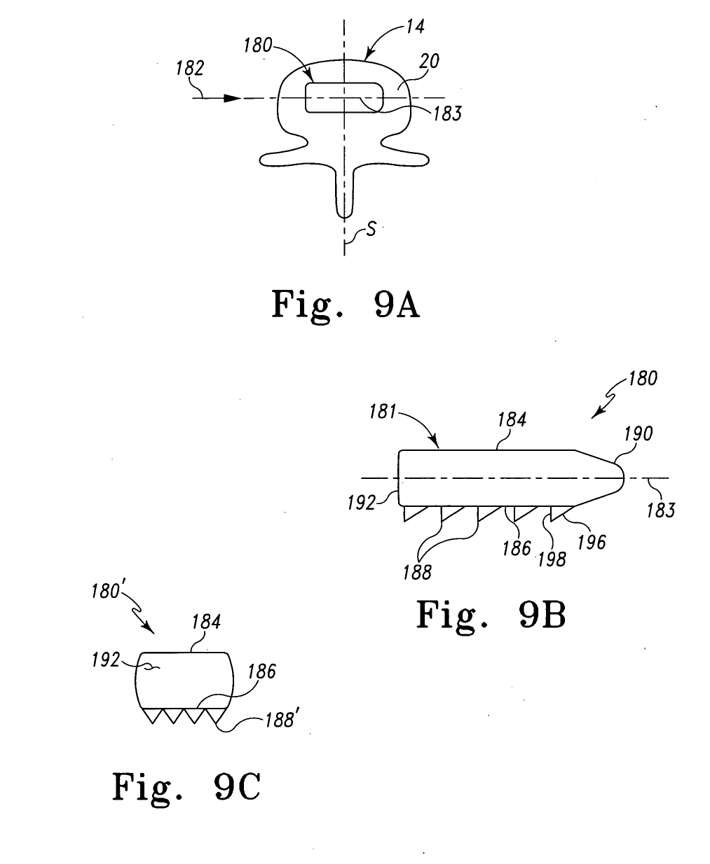

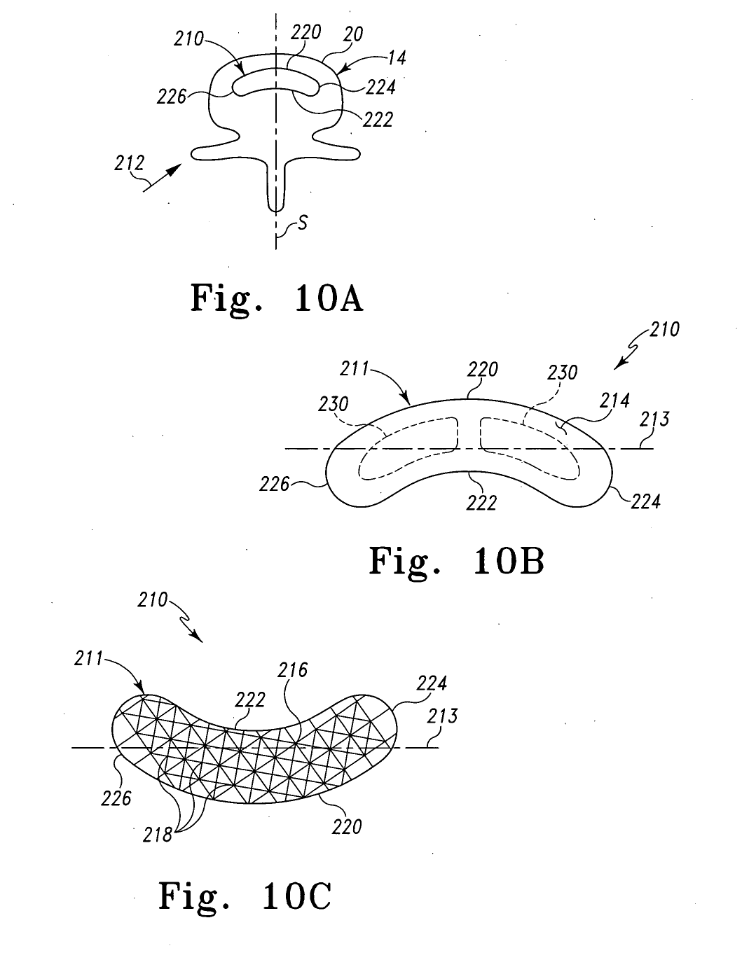

FIG. 9A shows inferior vertebra 14 and disc space 20 with another embodiment of spinal implant 30, designated as spinal implant 180, positioned in disc space 20. Spinal implant 180 includes a size and shape that occupies a portion of disc space 20 and supports the adjacent vertebrae when positioned in disc space 20 from a lateral approach to disc space 20, as indicated by arrow 182. Spinal implant 180 includes an elongated body 181 extending along a longitudinal axis 183 aligned orthogonally to sagittal plane S in the implanted orientation.

As also shown in FIG. 9B, body 181 includes a distal leading end nose 190 and a proximal trailing end 192. Superior bearing surface 184 extends along body 181 and provides a smooth surface along which superior vertebra 12 can be moved. Inferior bearing surface 186 is located opposite superior bearing surface 184, and includes engaging features 188 extending therefrom.

Engaging features 188 are illustrated as elongated ridges that extend transversely to longitudinal axis 183 so that the ridges extend anteriorly-posteriorly when positioned in disc space 20. The ridges are shown with a ratcheting configuration where the leading side 196 is sloped to facilitate insertion and the trailing side 198 is more vertically oriented relative to inferior bearing surface 186 to resist movement in direction opposite the insertion direction.

In FIG. 9C, an end elevation view of another embodiment spinal implant 180′ is shown that is the same as spinal implant 180 except the engaging features 188′ are oriented orthogonally to the direction of FIG. 9B. When spinal implant 180′ is implanted, the ridges extend in the medial-lateral direction. Other embodiments contemplate engaging features in the form of teeth so that resistance to movement of spinal implant 180 relative to inferior vertebra 14 is the same in all directions. Still other embodiments contemplate engaging features and insertional tool engaging features as described herein with respect to the other embodiments.

Referring now to FIG. 10A, there is shown inferior vertebra 14 and disc space 20 with another embodiment of spinal implant 30, designated as spinal implant 210, positioned in disc space 20. Spinal implant 210 includes a size and shape that occupies an anterior portion of disc space 20 while providing bi-lateral support of the adjacent vertebrae when positioned in disc space 20 from a postero-lateral approach to disc space 20, as indicated by arrow 212. As also shown in FIGS. 10B and 10C, spinal implant 210 includes an elongated body 211 extending along a longitudinal axis 213 that forms a concave-convex profile along longitudinal axis 213. Body 211 includes an anterior wall or surface 220 that is convexly curved and a posterior wall or surface 222 that is concavely curved. Leading and trailing ends 224, 226 extend between anterior and posterior surfaces 220, 222 to form a convexly curved ends of body 211.

Body 211 further includes superior bearing surface 214 for contacting the inferior surface of superior vertebra 12 and opposite inferior bearing surface 216 for contacting the superior surface of inferior vertebra 14. Superior bearing surface 214 is smooth to facilitate movement of superior vertebra 12 therealong, while inferior bearing surface 216 includes engaging features such as spikes 218 to engage inferior vertebra 14 and prevent movement of spinal implant 210 relative to inferior vertebra 14 while the superior vertebra 12 is moved into alignment.

Spikes 218 cover substantially all the entire surface area of inferior bearing surface 216. Other embodiments contemplate spikes that cover less than all the surface area of inferior bearing surface 216. Other embodiments contemplate other arrangements for the engaging features, including any of the engaging feature arrangements discussed herein for the other embodiment implants. Body 211 is shown as solid. In other embodiments, one or more cavities 230, as indicated in dashed lines in FIG. 10B, can be provided that open through bearing surfaces 214, 216. Body 211 can also be provided with insertion tool engaging at or adjacent one or both of ends 224, 226 to facilitate engagement with an insertion tool.

In other embodiments, the spinal implant is configured for insertion into the disc space from an antero-lateral approach. The superior bearing surface of the implant is smooth, while the inferior bearing surface includes engaging features to engage the inferior vertebrae when implanted.

In other procedures it may be desired to re-position or align the inferior vertebra. Therefore, the spinal implants discussed herein can be arranged with a smooth profile along their inferior bearing surface and engaging features extending from the superior bearing surface. The engaging features positively engage the superior vertebra to maintain the implant positioning relative to the superior vertebra while the inferior vertebra is moved along the smooth inferior bearing surface into the desired position.

The spinal implants discussed herein can be made from any suitable biocompatible material, including bone material, metals and metal alloys, polymers and polymer composites, carbon fiber material, ceramics, and combinations of various materials. The materials can be non-resorbable, or resorbable over time. In another embodiment, the smooth bearing surface is provided with a surface coating or layer of lubricious or low friction material that facilitates sliding movement of the vertebra along the smooth bearing surface.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A spinal implant, comprising:
a body sized for positioning in a spinal disc space between adjacent vertebrae, said body including a first bearing surface and an opposite second bearing surface extending along said body, said body further including a height between said first and second bearing surfaces that provides a restored disc space height when positioned in the spinal disc space, wherein said first and second bearing surfaces define a respective entire side of said body and said sides are positionable in contact with respective ones of the adjacent vertebrae and said first bearing surface is entirely smooth and said second bearing surface includes means for engaging bony material to secure said body in position with the respective one of the adjacent vertebrae.

2. The implant of claim 1, wherein said first and second bearing surfaces are convexly curved.

3. The implant of claim 2, wherein said body comprises:
opposite sidewalls extending along a length of said body and between said first and second bearing surfaces; and
a leading end nose connecting said opposite sidewalls and said first and second bearing surfaces.

4. The implant of claim 3, wherein said leading end nose is convexly curved between said opposite sidewalls and convexly curved between said first and second bearing surfaces.

5. The implant of claim 1, wherein said body includes a cavity extending therethrough opening through each of said first and second bearing surfaces.

6. The implant of claim 5, further comprising bone growth material in said cavity.

7. The implant of claim 1, wherein said means for engaging bony material include a number of teeth extending from said second bearing surface, said teeth being located between a cavity opening through said second bearing surface and an adjacent sidewall of said body.

8. The implant of claim 7, wherein said teeth are V-shaped and grooves are provided between adjacent ones of said teeth.

9. The implant of claim 1, wherein said means for engaging bony material are formed by a number of grooves extending across said second bearing surface.

10. The implant of claim 1, wherein said body includes opposite sidewalls extending between said first and second bearing surfaces, said sidewalls forming a convexly curved shape between said first and second bearing surfaces.

11. The implant of claim 10, wherein said means for engaging include threads extending from said opposite sidewalls and said second bearing surface.

12. The implant of claim 1, wherein when implanted in the disc space said body defines a rectangular shape when viewed in the axial plane of the spinal column.

13. The implant of claim 1, wherein when implanted in the disc space said body defines a D-shape shape when viewed in the axial plane of the spinal column.

14. The implant of claim 1, wherein when implanted in the disc space said body defines a shape including a concavely curved posterior wall surface and a convexly curved anterior wall surface when viewed in the axial plane of the spinal column.

15. The implant of claim 1, wherein said body is configured so when implanted in the disc space said first bearing surface is positioned along an endplate of a superior one of the adjacent vertebrae and said second bearing surface is positioned along an endplate of an inferior one of the adjacent vertebrae.

16. A method for correcting alignment of a spinal column, comprising:
positioning a spinal implant in a disc space between first and second vertebrae;
fixing the spinal implant in position with the first vertebra; and
sliding the second vertebra along a smooth bearing surface of the spinal implant to align the first and second vertebrae.

17. The method of claim 16, wherein the first vertebra is an inferior vertebra and the second vertebra is a superior vertebra.

18. The method of claim 17, wherein fixing the spinal implant includes extending engagement means projecting from a second bearing surface of the implant into bone of the first vertebra.

19. The method of claim 18, wherein the engagement means includes a number of teeth extending from the second bearing surface.

20. The method of claim 18, wherein sliding the second vertebra includes sliding the second vertebra while maintaining contact between the smooth bearing surface and an endplate of the second vertebra.

21. The method of claim 16, wherein positioning the spinal implant includes positioning the spinal implant in the disc space from an anterior approach to the disc space.

22. The method of claim 16, wherein positioning the spinal implant includes positioning the spinal implant in the disc space from a lateral approach to the disc space.

23. The method of claim 16, wherein positioning the spinal implant includes positioning the spinal implant in the disc space from a postero-lateral approach to the disc space.

24. The method of claim 16, wherein positioning the spinal implant includes positioning the spinal implant in the disc space from a posterior approach to the disc space.

25. The method of claim 24, further comprising:
positioning a second spinal implant in the disc space between first and second vertebrae;
fixing the second spinal implant in position with the first vertebra; and
sliding the second vertebra includes sliding the second vertebra along smooth bearing surfaces of each of the spinal implants to align the first and second vertebrae.

26. The method of claim 16, further comprising engaging a spinal stabilization construct between the first and second vertebrae, wherein the stabilization construct includes first and second anchors engaged to respective ones of the first and second vertebrae and an elongated stabilization element engaged between the first and second anchors.

27. The method of claim 16, wherein positioning the spinal implant includes recapitulating the disc space with a rounded leading end nose extending between the smooth bearing surface and an opposite second bearing surface fixed to the first vertebra.

29. A spinal implant, comprising:
a body sized for positioning in a spinal disc space between adjacent vertebrae, said body extending along an axis between a distal leading end and a proximal trailing end, said body further including sidewalls extending between said leading end and said trailing end, wherein said body further includes:
a first bearing surface extending along a first side of said body between said sidewalls and said leading end and said trailing end, wherein said first bearing surface defines an entirely smooth surface profile for contacting one of the adjacent vertebrae; and
a second bearing surface extending along a second side of said body between said sidewalls and said leading end and said trailing end, wherein said second bearing surface includes engaging features extending therefrom for engaging the other of the adjacent vertebrae.

30. The implant of claim 29, wherein said sidewalls are parallel.

31. The implant of claim 30, wherein said body is elongated and said axis is a longitudinal axis.

32. The implant of claim 31, wherein said leading end forms a rounded nose convexly curved between said opposite sidewalls and convexly curved between said first and second bearing surfaces.

33. The implant of claim 29, wherein said body includes a cavity extending therethrough and opening at each of said first and second bearing surfaces.

34. The implant of claim 29, wherein said leading end includes a convexly curved wall extending between said sidewalls.

35. The implant of claim 34, wherein said trailing end includes a concavely curved wall extending between said sidewalls.

36. The implant of claim 35, wherein said sidewalls are convexly curved between said convexly curved wall at said leading end and said concavely curved wall at said trailing end.

37. The implant of claim 34, wherein said sidewalls are parallel and said trailing end includes a linear wall extending between said sidewalls.

38. The implant of claim 29, wherein said first and second bearing surfaces are convexly curved.

39. The implant of claim 29, wherein said first and second bearing surfaces are parallel to one another.

40. The implant of claim 29, wherein said body defines a first height between said first and second bearing surfaces adjacent said distal leading end that is different than a second height defined between said first and second bearing surfaces adjacent said proximal trailing end.
